(19) United States
(12) Patent Application Publication
(10) Pub. No.: US 2025/0120631 A1
(43) Pub. Date: Apr. 17, 2025

(54) APPARATUS AND METHOD FOR DIAGNOSING DISEASE BASED ON IMAGE

(71) Applicant: Electronics and Telecommunications Research Institute, Daejeon (KR)

(72) Inventors: Mun Seob LEE, Daejeon (KR); Sang Yun KIM, Daejeon (KR); Chi Hoon KIM, Daejeon (KR); Jong Jin LEE, Daejeon (KR)

(73) Assignee: Electronics and Telecommunications Research Institute, Daejeon (KR)

(21) Appl. No.: 18/821,503

(22) Filed: Aug. 30, 2024

(30) Foreign Application Priority Data
Oct. 13, 2023 (KR) 10-2023-0136598
Jun. 12, 2024 (KR) 10-2024-0076495

(51) Int. Cl. A61B 5/16; A61B 5/00; G06T 11/00; G06V 10/764; G06V 10/774; G06V 10/82; G06V 20/40; G06V 40/16; G06V 40/20; G10L 15/08; G10L 15/16; G10L 25/18; G10L 25/57; G10L 25/63; G10L 25/66; G16H 50/20

(52) U.S. Cl. CPC A61B 5/165; A61B 5/4803; G06T 11/001; G06V 10/764; G06V 10/774; G06V 10/82; G06V 20/41; G10L 15/16; G10L 25/18; G10L 25/57; G10L 25/63; G10L 25/66; G16H 50/20; G06V 40/174; G06V 40/20; G06V 2201/03; G10L 2015/088

(57) ABSTRACT

Provided is an apparatus for diagnosing a disease, which includes an acquisition module configured to acquire multi-modal data including at least two types of data among text data, speech data, and image data related to each depression patient, a preprocessing module configured to visualize data that is not the image data among the multi-modal data and output image datasets including the image data among the multi-modal data and the visualized data, and a classification module configured to classify whether each depression patient has depression based on the image datasets.

### CROSS-REFERENCE TO RELATED APPLICATION

This application claims priority to and the benefit of Korean Patent Applications No. 10-2023-0136598, filed on Oct. 13, 2023, and No. 10-2024-0076495, filed on Jun. 12, 2024, the disclosure of which is incorporated herein by reference in its entirety.

### BACKGROUND

### 1. Field of the Invention

Various embodiments disclosed in this document relate to an artificial intelligence (AI) model for diagnosing a disease based on an image.

### 2. Discussion of Related Art

Depression, referred to as the cold of the mind, is a mental disease that affects approximately 280 million people worldwide. Depression having an attack rate of approximately 3.8%, is a mental disease with a significant social and economic burden, and has a significant impact on individuals' quality of life. Since the outbreak of COVID-19 pandemic, the attack rate of depression has risen to about 25%, and an annual average of more than one million people in Korea is diagnosed with depression.

The diagnosis of depression is performed in various methods, each method having its own advantages and disadvantages. First, there is a clinic identification method that uses expert questionnaires, or expert questionnaires together with medical devices, such as functional magnetic resonance imaging (fMRI) and computer tomography (CT). Second, there is a method of using depression screening tools through questionnaires, such as PHQ-9, BDI, and CESD-R, but this method not only takes a long time for medical staff, but also lacks accuracy and reliability in the depression diagnosis. Third, there is a method of identifying the functional and physiological characteristics of the brain using fMRI or CT, but this method may be costly and have a difficulty in early diagnosis.

### SUMMARY OF THE INVENTION

Recently, research has been conducted to diagnose psychical disorders (e.g., depression) by collecting data using information and communication technology (ICT) and training an artificial intelligence (AI) models, such as a deep learning AI model, with the collected data.

FIG. 1 is an exemplary diagram illustrating an AI model related to diagnosis of depression.

Referring to FIG. 1, collected data is largely classified into i) behavioral data of patients, such as text, videos, and speeches, ii) social data of patients, such as social networking services (SNSs) and Instagram, and iii) physiological data on anxiety symptoms, such as eye movements, heart rates, and behavioral patterns.

An apparatus for diagnosing depression may use the collected data, including single data or multi-modal data, to diagnose depression through an AI model. The collected data has different dimensions and different data types and characteristics. Therefore, the apparatus for diagnosing depression may, in order to process different types of data, generate separate AI models and combine diagnosis results of each AI model using an ensemble technique to diagnose/evaluate depression. However, such an apparatus for diagnosing depression requires a large AI network and capacity, consuming a great amount of time or energy. Additionally, due to inconsistencies in the learning results between different types of data, the processing accuracy may decrease.

Various embodiments disclosed in this document may provide an apparatus and method for diagnosing a disease being capable of identifying the presence of depression based on image data which is visualized input data.

According to an aspect of the present invention, there is provided an apparatus for diagnosing a disease, which includes an acquisition module configured to acquire multi-modal data including at least two types of data among text data, speech data, and image data related to a test subject, a preprocessing module configured to visualize data that is not the image data among the multi-modal data and output image datasets including the image data and the visualized data; and a classification module configured to classify whether the test subject has a specified disease based on the image datasets.

According to an aspect of the present invention, there is provided a method of diagnosing a disease, which is performed by at least one processor, the method including acquiring multi-modal data including at least two types of data among text data, speech data, and image data related to a test subject, visualizing data that is not the image data among the multi-modal data, outputting image datasets including the image data and the visualized data, and classifying whether the test subject has a specified disease based on the image datasets.

According to an aspect of the present invention, there is provided an apparatus for diagnosing a disease, including a memory including at least one instruction, and a processor functionally connected to the memory, wherein when executed, the at least one instruction causes the processor to: acquire multi-modal data including at least two types of data among text data, speech data, and image data related to a test subject, visualize data that is not the image data among the multi-modal data, and output an image dataset including the image data and the visualized data, and classify whether the test subject has a specified disease based on the image dataset.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is an exemplary diagram illustrating an artificial intelligence (AI) model related to depression diagnosis;

FIG. 2 is a conceptual diagram illustrating an apparatus for diagnosing a disease according to an embodiment;

FIG. 3 is a block diagram illustrating a configuration of the apparatus for diagnosing a disease according to the embodiment;

FIG. 4 is an exemplary diagram illustrating a word cloud according to an embodiment;

FIG. 5 is an exemplary diagram illustrating a Mel spectrogram generated based on 10 minutes speech data of a depression patient according to an embodiment;

FIG. 6 illustrates an example of an image dataset according to an embodiment;

FIG. 7 is a block diagram illustrating a detailed configuration of a classification module according to an embodiment;

FIG. 8 is a diagram illustrating a training process of a classification module according to an embodiment; and

FIG. 9 is a flowchart showing a method of diagnosing a disease according to an embodiment.

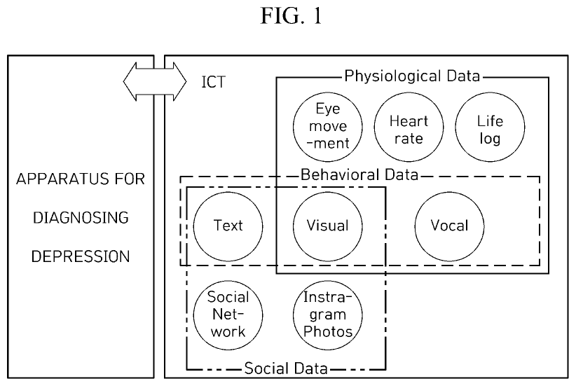

In relation to the description of the drawings, identical or similar reference numerals may be used for identical or similar components

### DETAILED DESCRIPTION OF EXEMPLARY EMBODIMENTS

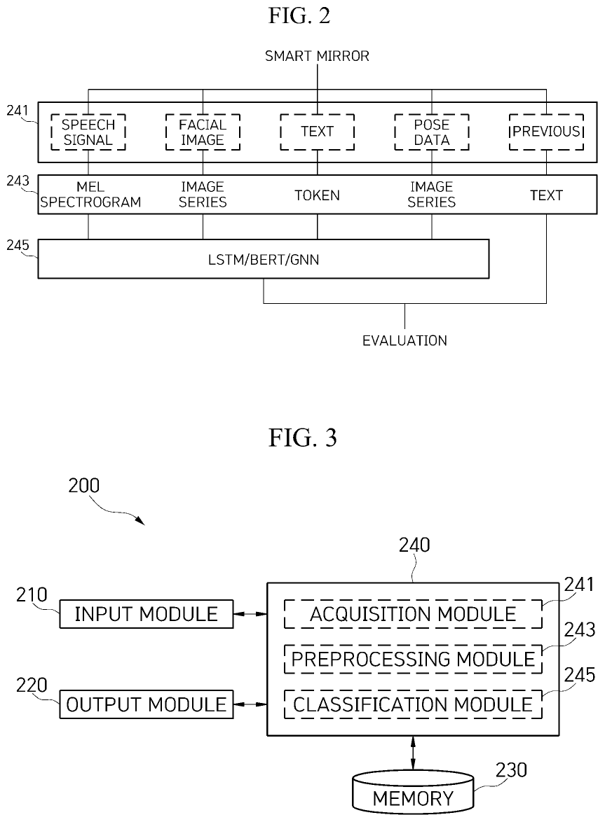

FIG. 2 is a conceptual diagram illustrating an apparatus for diagnosing a disease according to an embodiment, and FIG. 3 is a block diagram illustrating a configuration of the apparatus for diagnosing a disease according to the embodiment.

Referring to FIG. 2, an apparatus 200 for diagnosing a disease according to an embodiment may acquire multi-modal data including at least two types of data among speech data, image data, and text data related to test subjects. The test subjects may include a patient having a specified disease (e.g., a depression patient) and normal people. The multi-modal data may be generated from a video obtained during a process of diagnosing a specified disease for a test subject. The image data may include a facial image including a face area of a user and pose data including a body pose (or a movement) of a user. The specified disease may include at least one of depression, bipolar disorder, anxiety, depressive disorder, or anxiety disorder.

According to an embodiment, the apparatus 200 for diagnosing a disease may visualize each piece of data that is not image data included in the multi-modal data. For example, the apparatus 200 for diagnosing a disease may visualize speech data using a Mel spectrogram technique and visualize text as a word cloud based on emotional keywords.

According to an embodiment, the apparatus 200 for diagnosing a disease may identify (or classify) whether a test subject has a specified disease by learning each visualized image data with a network model of two or more dimensions.

Referring to FIG. 3, the apparatus 200 for diagnosing a disease according to an embodiment may include an input module 210, an output module 220, a memory 230, and a processor 240. In an embodiment, in the apparatus 200 for diagnosing a disease, some components may be omitted, or additional components may be added. Additionally, some of the components of the apparatus 200 for diagnosing a disease may be combined into a single component but perform the same functions of the components before the combination.

The input module 210 may receive user input using the apparatus 200 for diagnosing a disease. The input module 210 may include at least one input detection circuit of, for example, a touch screen, a microphone, a camera, a keyboard, and a mouse. The input module 210 may include a communication module or may be a communication module. For example, the input module 210 may be a microphone that recognizes or detects a user's speech. For another example, the input module 210 may be a camera that photographs a user's face or body. As another example, the input module 210 may be a keyboard or a touch screen that receives user's text. Alternatively, the input module 210 may be a communication module that acquires (e.g., copies and stores) text written by a user from social networking services.

The output module 220 may visually or audibly output at least one type of data among symbols, numbers, or letters under the control of the processor 240. The output module 220 may include at least one output device among, for example, a liquid crystal display, an organic light emitting diode (OLED), a touch screen display, and a speaker. For example, the output module 220 may output text or a speech indicating whether the test subject has a specified disease.

The memory 230 may include various types of volatile memories or non-volatile memories. For example, the memory 230 may include a read only memory (ROM) and a random access memory (RAM). In an embodiment, the memory 230 may be located inside or outside the processor 240, and the memory 230 may be connected to the processor 240 through various known means. The memory 230 may store various types of data used by at least one component (e.g., the processor 240) of the apparatus 200 for diagnosing a disease. The data may include, for example, software and input data or output data regarding instructions related thereto. For example, the memory 230 may store at least one instruction and data for providing a disease diagnosis service. When executed, the at least one instruction may cause the processor 240 to: acquire multi-modal data including at least two types of data among text data, speech data, and image data related to a test subject; visualize data that is not the image data among the multi-modal data, and output an image dataset including the image data and the visualized data; and classify whether the test subject has a specified disease (e.g., a depression patient or a normal person) based on the image dataset.

According to an embodiment, the memory 230 may include data related to a conversion of text data to a word cloud, for example, an emotional evaluation dictionary. For example, the emotional evaluation dictionary may include scores related to the emotional meaning of each word. The emotional evaluation dictionary may include, for example, affective norms for English words (ANEW) database.

According to an embodiment, the memory 230 may store instructions and data related to image and speech segmentation of a video, conversion of speech to text, visualization of speech data and text data, or determination of the presence of a specified disease based on an image dataset. The memory 230 may further store instructions and data related to lightening image data or augmenting image data.

The processor 240 may control at least one other component (e.g., a hardware component or a software component) of the apparatus 200 for diagnosing a disease and may perform various data processing processes or calculations. For example, the processor 240 may include at least one of a central processing unit (CPU), a graphics processing unit (GPU), a microprocessor, an application processor, an application specific integrated circuit (ASIC), and a field programmable gate array (FPGA), and may have a plurality of cores.

According to an embodiment, the processor 240 may include an acquisition module 241, a preprocessing module 243, and a classification module 245. The acquisition module 241, the preprocessing module 243, and the classification module 245 may be a software module or a hardware module included in the processor 240 or executed by the processor 240.

According to an embodiment, the acquisition module 241 may acquire multi-modal data including at least two types of data among image data, speech data, and text data related to test subjects (users). The acquisition module 241 may generate image data, speech data, and text data based on videos recorded (audio and video) during a diagnosis process of each user (test subject). The image data may be a video image that includes user's facial expressions and body movements recorded during the diagnosis process. The speech data may be a result of digital conversion of user's speeches recorded during the diagnosis process. The text data may be data obtained by converting writing or speech recorded during the diagnosis process into text. Additionally, the text data may further include data recorded on the user's social networking services.

In this regard, the processor 240 may receive (or acquire) video data of the user (test subject) who participates in the diagnosis process through the input module 210. Additionally, the processor 240 may further acquire previously written past text data from the user's social networking services through the input module 210. The processor 240 may acquire multi-modal data from the video data and the past text data acquired through the acquisition module 241.

According to an embodiment, the preprocessing module 243 may visualize data that is not image data, such as text data and speech data, among the multi-modal data.

According to an embodiment, the preprocessing module 243 may extract specified emotional keywords (words) from the text data. The preprocessing module 243 may tokenize the emotional keywords extracted from the text data and convert the tokens into images.

For example, the preprocessing module 243 may extract pronouns, nouns, and predicates excluding suffixes and word phrases other than the extracted emotional keywords. The preprocessing module 243 may check the frequencies of appearance of the extracted emotional keywords (nouns, pronouns, and predicates) and the scores of the extracted emotional keywords (nouns, pronouns, and predicates) according to the emotional evaluation dictionary. The preprocessing module 243 may generate a word cloud representing the extracted emotional keywords to correspond to the frequency of appearance and the checked score. For example, the preprocessing module 243 may express the keyword in a larger size and darker color as the frequency of appearance and the checked score increase.

According to an embodiment, the preprocessing module 243 may divide the time-domain speech data for each frame (or each specified time interval) and perform fast Fourier transform (FFT), thereby converting the time-domain speech data into the frequency-domain speech data. The preprocessing module 243 may apply a Mel spectrogram technique to each piece of frequency information (spectrum) in the frequency domain to calculate a Mel spectrogram. The Mel spectrogram technique is one of the techniques that mathematically converts frequency information by reflecting the human auditory structure (Mel scale), and may visually represent frequency changes over time. The horizontal axis of the Mel spectrogram represents temporal characteristics, and the vertical axis represents frequency. For example, the preprocessing module 243 may process the Mel spectrogram with a log scale to generate a log Mel spectrogram. For example, depression patients differ from normal people in tone, intensity, variation, and pauses of the speech (voice) data. In an embodiment, whether a patient has depression may be distinguished based on the differences represented on the Mel spectrogram.

According to an embodiment, the preprocessing module 243 may preprocess (e.g., lighten) image data in multi-modal data. For example, when the image data is color image data of two or more dimensions, the preprocessing module 243 may convert the image data into one-dimensional black and white image data. For another example, the preprocessing module 243 may convert time-series still images included in a video image into one-dimensional black and white images.

According to an embodiment, the preprocessing module 243 may output an interrelated image dataset including the preprocessed image data, the visualized speech data, and the visualized text data.

In an embodiment, the preprocessing module 243 may, when the number of image datasets is less than a predetermined number during the training process, augment the image datasets using a data augmentation technique or a k-fold training technique. The specified number may be experimentally determined as a number that may ensure the classification performance.

According to an embodiment, the classification module 245 may learn image data for diagnosis to classify whether a test subject has a specified disease. For example, the classification module 245 may include a three-dimensional single network model based on convolution. The network model may be, for example, one of a Convolution Neural Network (CNN), Transformer, Attention, Graph neural network, and Boltzmann network.

During the training process, the classification module 245 may receive the image dataset including the preprocessed image data, the visualized speech data, and the visualized text data. For example, the classification module 245 may acquire lightened image data (a one-dimensional black and white image), a Mel spectrogram, and a word cloud as input thereof.

The classification module 245 may be configured to classify whether a test subject is a patient of a specified disease by learning feature values of the image dataset for training. The feature values of the preprocessed image data may include, for example, feature values related to facial expressions and body poses, and the rate of change thereof. Feature values of the visualized speech data may include, for example, feature values related to a tone, an intensity, a variation, and a pause. Feature values of the visualized text data may include a size or a color for each word.

According to an embodiment, the processor 240 may output a result of the classifying (diagnosing) of the classification module 245 whether a specified disease is present through the output module 220. For example, the processor 240 may output diagnostic results to be distinguished between depression patients and normal people with respect to each image dataset. The specified disease may vary, but in the description of the present disclosure, depression is used as an example of the specified disease. However, the present invention is not limited thereto. For example, the specified disease may include at least one other disease beside depression, such as bipolar disorder, anxiety, depressive disorder, or anxiety disorder, or other psychical disorder (e.g., panic disorder).

In the above-described embodiment, the processor 240 divides all multi-modal data of each user for each certain time interval and performs training and classification. Accordingly, the processor 240 may aggregate the diagnosis results for the all multi-modal data of each user to classify (diagnose) whether each test subject has a specified disease.

According to various embodiments, the processor 240 may record a video of a user participating in a diagnosis process by a smart device and receive the user video from the smart device through the input module 210 (e.g., a communication module). The smart device may include a smart mirror, a smartphone, and a smart app. The processor 240 uses the smart device, which is interactive with users, enabling real-time diagnosis, emotional interaction with users, and provision of diagnosis results to medical staff.

According to various embodiments, the processor 240 may additionally utilize at least one type of data among metadata, a heart rate, health data, and a life log to diagnose whether a patient has a specified disease. In this case, the processor 240 may further include another classification module capable of diagnosing whether a specified disease (e.g., depression) is present based on at least one other type of data. And the processor 240 may diagnose whether the specified disease is present based on the other classification module. The processor 240 may aggregate classification results based on the classification module 245 and the other classification module and provide the classification results through the output module 220. For example, the processor 240 may provide all classification results of each classification module 245.

As described above, the apparatus 200 for diagnosing a disease according to an embodiment may visualize data obtained through the device to construct a training dataset or a diagnostic dataset, and input the visualized training dataset or the visualized diagnostic dataset into a single diagnostic network model to diagnose a specified psychical disorder. Accordingly, a single diagnosis network model having a significantly reduced size may be provided, and accuracy and diagnosis speed may be further improved.

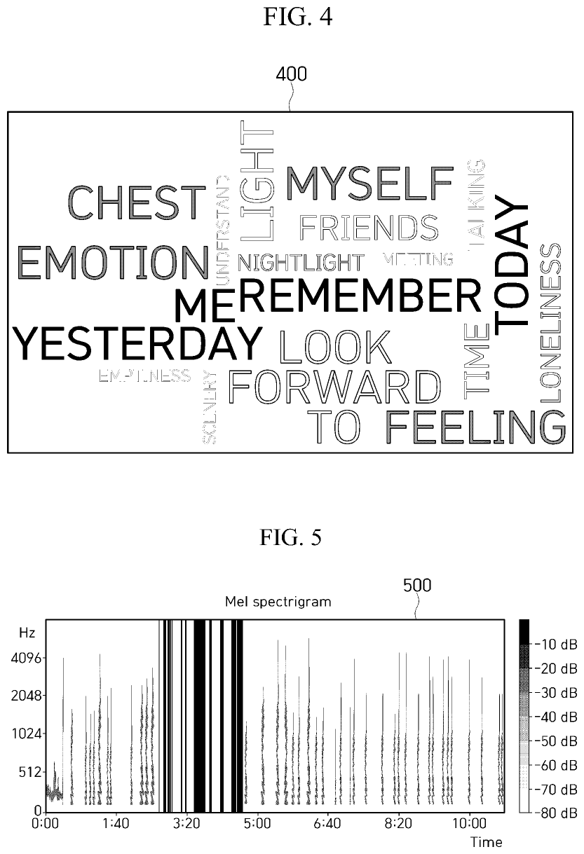

FIG. 4 is an exemplary diagram illustrating a word cloud according to an embodiment. In FIG. 4, an example of a word cloud expressed from text data generated based on a speech of a depression patient is illustrated.

These days I don't recognize myself. When I open my eyes in the
morning, that heavy feeling immediately presses down on my chest. It
feels like today will be just like yesterday. I can't even remember
yesterday properly, so it is too hard to tell me to look forward to today.
I don't know what I should get up for, why I should smile, or why I
should cry. All my emotions are blurry. Like the light of a nightlight
coming from far away. People tell me I need time to get better. But will
time really heal me? I feel like I'll still be standing in the same place
even after time has passed.
Everything such as meeting with friends and talking with family should
feel so important and precious, but to me, it feels like just passing
scenery. Is there anyone who truly understands me? Or can I understand
myself?
When will this emptiness and loneliness I feel disappear? Or is this the
real me? Who on earth am I?

Referring to FIG. 4, the apparatus 200 for diagnosing a disease according to the embodiment may extract all emotional keywords (pronouns, nouns, and predicates) included in the text data. The apparatus 200 for diagnosing a disease may nominalize predicates among the extracted emotional keywords and match the emotional keywords with the frequency of appearance and the emotion score according to the emotion evaluation dictionary corresponding thereto. The apparatus 200 for diagnosing a disease may generate a word cloud that displays each extracted emotional keyword in a color and a size based on the frequency of appearance and the emotional score. In the word cloud shown in FIG. 4, emotional keywords, such as “chest,” “emotion,” “me,” “remember,” “look forward to,” “yesterday,” “feeling,” “today,” and “myself” may have a relatively lower frequency or emotion score than emotional keywords, such as “light,” “family,” “nightlight,” “understand,” “scenery,” “emptiness,” “meeting,” “friend,” “time,” “talking,” and “loneliness.”

FIG. 5 is an exemplary diagram illustrating a Mel spectrogram generated based on 10 minutes speech data of a depression patient according to an embodiment.

Referring to FIG. 5, the preprocessing module 243 according to the embodiment may represent the frequency magnitude over time through a Mel spectrogram. Accordingly, the apparatus 200 for diagnosing a disease may extract image feature values related to a tone, an intensity, a variation, and a pause from the Mel spectrogram, and classify whether depression is present based on the extracted feature values (or feature vectors).

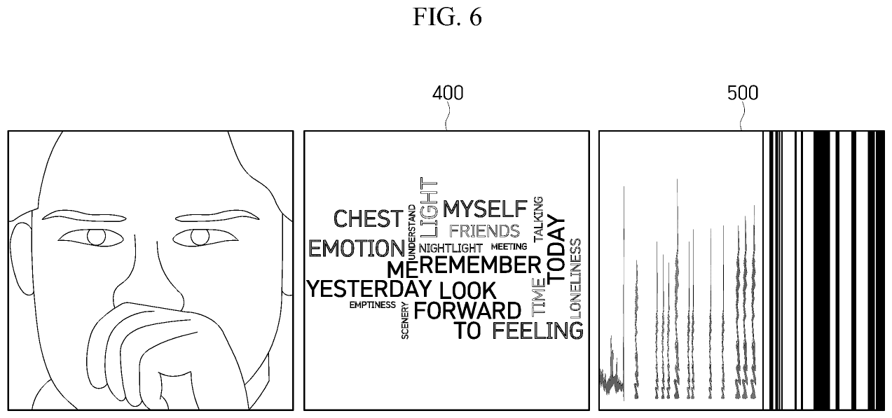

FIG. 6 illustrates an example of an image dataset according to an embodiment.

Referring to FIG. 6, the image dataset may include visualized text data, visualized speech data, and facial images and pose images (images including body poses). The visualized text data may be a word cloud image of tokenized text data of a depression patient. The visualized speech data may be an image of speech data converted into a Mel spectrogram. In an embodiment, the image dataset may be divided based on sentences (the points in time of a start and an end of a word phrase or a paragraph) recorded or uttered by the user (test subject) and trained (or tested). Alternatively, the image dataset may include a dataset of a depression patient acquired for each certain time interval.

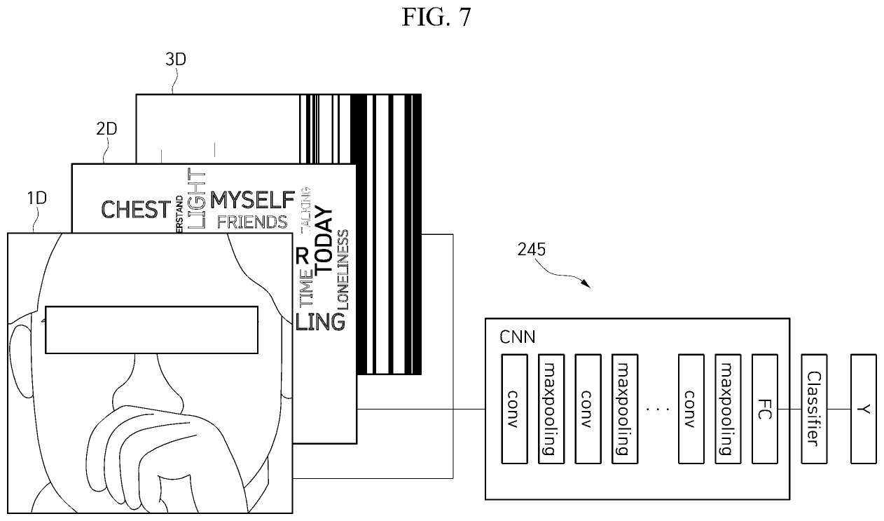

FIG. 7 is a block diagram illustrating a detailed configuration of a classification module according to an embodiment.

Referring to FIG. 7, the classification module 245 according to an embodiment may include a 3D single network model including a convolutional layer conv and a maxpooling layer. The classification module 245 may learn the input image dataset using the 3D single network model. During the training process, the classification module 245 may learn labels of the image dataset that may distinguish depression patients and normal people for each image dataset with respect to feature values of the image dataset.

As described above, the related art of AI model diagnoses depression using an ensemble technique using data with different dimensions, different types, and characteristics. Alternatively, the classification module 245 according to the embodiment may classify whether depression is present using a single network based on image data, which allows for a reduction in the size of the network model and construction of an optimized learning model.

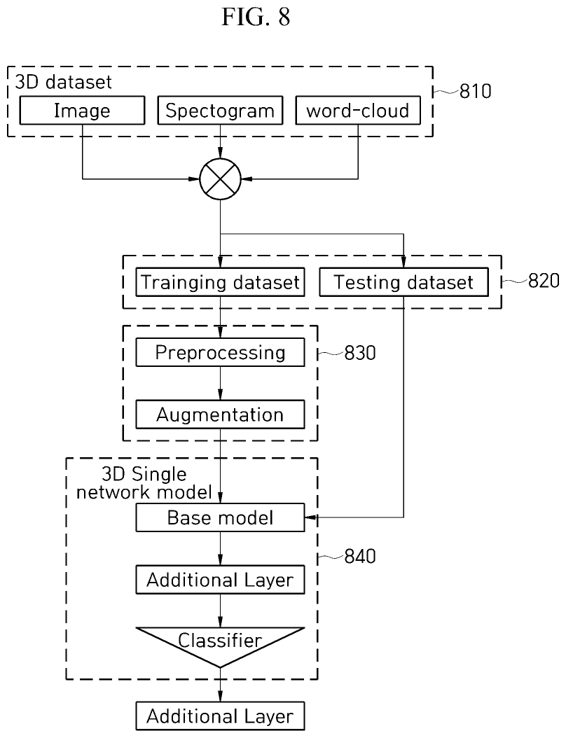

FIG. 8 is a diagram illustrating a training process of a classification module 245 according to an embodiment.

Referring to FIG. 8, in operation 810, the apparatus 200 for diagnosing a disease may acquire a three-dimensional image dataset including preprocessed image data, visualized speech characteristics (a spectrogram), and visualized text data (a word cloud).

In operation 820, the apparatus 200 for diagnosing a disease may divide the acquired 3D image dataset into a part and the remaining part as a training dataset and a test dataset, respectively.

In operation 830, the apparatus 200 for diagnosing a disease may perform additional preprocessing to improve the accuracy of training. For example, the apparatus 200 for diagnosing a disease may increase the number of pieces of data using a data augmentation technique or a k-fold training techniques with respect to a small number of training datasets.

In operation 840, the apparatus 200 for diagnosing a disease inputs the training dataset into the classification module 245 and trains the network through a classifier.

Thereafter, the apparatus 200 for diagnosing a disease may diagnose whether a depression patient has depression using the trained classification module 245 using the test dataset.

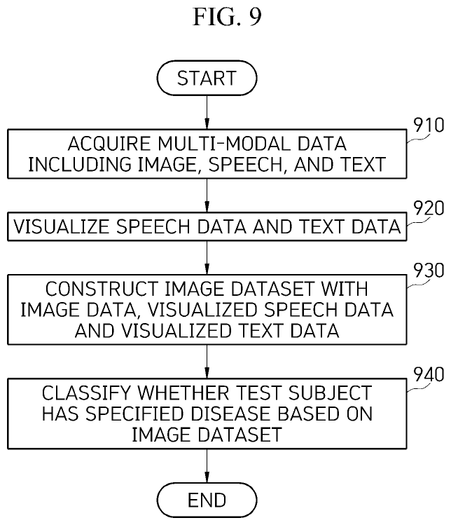

FIG. 9 is a flowchart showing a method of diagnosing a disease according to an embodiment.

Referring to FIG. 9, in operation 910, the apparatus 200 for diagnosing a disease may acquire multi-modal data including at least two types of data among text data, speech data, and image data related to a test subject (e.g., a depressed patient). For example, the apparatus 200 for diagnosing a disease may extract each of the image data and the speech data from video data recorded during a diagnosis of the test subject. The image data may include sequential image frames related to at least one of facial expressions or body movements. For another example, the apparatus 200 for diagnosing a disease may convert the speech data extracted from the video data into text to generate text data. Additionally, or alternatively, the apparatus 200 for diagnosing a disease may extract the text data from social networking services of the test subject.

In operation 920, the apparatus 200 for diagnosing a disease may visualize data that is not image data among the multi-modal data. For example, the apparatus 200 for diagnosing a disease may extract emotional keywords from the text data and visualize the extracted emotional keywords as a word cloud. For another example, the apparatus 200 for diagnosing a disease may visualize the speech data by applying a Mel spectrogram technique to the speech data.

In operation 930, the apparatus 200 for diagnosing a disease may construct an image dataset including the image data among the multi-modal data and the visualized data. In operation 930, when the image data is color image data of two or more dimensions, the apparatus 200 for diagnosing a disease may convert the image data into one-dimensional black and white image data to lighten the image data.

In operation 940, the apparatus 200 for diagnosing a disease may classify whether the test subject has a specified disease based on the image dataset. The specified disease may include, for example, at least one of depression, bipolar disorder, anxiety, depressive disorder, or anxiety disorder. For example, the apparatus 200 for diagnosing a disease may classify whether the depressed patient has depression through a three-dimensional single network model with a convolutional structure.

As described above, the apparatus 200 for diagnosing a disease according to an embodiment is related to a method of diagnosing a disease including visualizing data obtained through the device to construct a training dataset or a diagnostic dataset, and inputting the visualized training dataset or the visualized diagnostic dataset into a single diagnostic network model to diagnose a specified psychical disorder. Accordingly, the method may provide a single diagnosis network model with a significantly reduced size, and may improve the accuracy and diagnosis speed.

The various embodiments of the disclosure and terminology used herein are not intended to limit the technical features of the disclosure to the specific embodiments, but rather should be understood to cover all modifications, equivalents, and alternatives falling within the spirit and scope of the invention. Like numbers refer to like elements throughout the description of the drawings. The singular forms preceded by “a,” “an,” and “the” corresponding to an item are intended to include the plural forms as well unless the context clearly indicates otherwise. In the disclosure, a phrase such as “A or B,” “at least one of A and B,” “at least one of A or B,” “A, B or C,” “at least one of A, B and C,” and “at least one of A, B, or C” may include any one of the items listed together in the corresponding phrase, or any possible combination thereof. Terms such as “first,” “second,” etc. are used to distinguish one element from another and do not modify the elements in other aspects (e.g., importance or sequence). When one (e.g., a first) element is referred to as being “coupled” or “connected” to another (e.g., a second) element with or without the term “functionally” or “communicatively,” it means that the one element is connected to the other element directly (e.g., by wire), wirelessly, or via a third element.

As used herein, the term “module” may include units implemented in hardware, software, or firmware, and may be interchangeably used with terms such as “logic,” “logic block,” “component,” or “circuit.” The module may be an integrally configured component or a minimum unit or part of the integrally configured component that performs one or more functions. For example, according to one embodiment, the module may be implemented in the form of an application-specific integrated circuit (ASIC).

The various embodiments of the present disclosure may be realized by software (e.g., a program) including one or more instructions stored in a storage medium (e.g., the database 140, such as an internal memory or external memory,) that can be read by a machine (e.g., the apparatus 100 for power grid simulation). For example, a processor (e.g., the processor 710) of the machine (e.g., the apparatus 100 for power grid simulation) may invoke and execute at least one instruction among the stored one or more instructions from the storage medium. Accordingly, the machine operates to perform at least one function in accordance with the invoked at least one command. The one or more instructions may include codes generated by a compiler or codes executable by an interpreter. The machine-readable storage medium may be provided in the form of a non-transitory storage medium. Here, when a storage medium is referred to as “non-transitory,” it can be understood that the storage medium is tangible and does not include a signal (for example, electromagnetic waves), but rather that data is semi-permanently or temporarily stored in the storage medium.

According to one embodiment, the methods according to the various embodiments disclosed herein may be provided in a computer program product. The computer program product may be traded between a seller and a buyer as a product. The computer program product may be distributed in the form of a machine-readable storage medium (e.g., a compact disc read only memory (CD-ROM)), or may be distributed directly between two user devices (e.g., smartphones) through an application store (e.g., Play Store™), or online (e.g., downloaded or uploaded). In the case of online distribution, at least a portion of the computer program product may be stored at least semi-permanently or may be temporarily generated in a machine-readable storage medium, such as a memory of a server of a manufacturer, a server of an application store, or a relay server.

Components according to various embodiments of the disclosure 0 may be implemented in the form of software or hardware, such as a digital signal processor (DSP), a FPGA or an ASIC, and may perform predetermined functions. The “elements” are not limited to meaning software or hardware. Each of the elements may be configured to be stored in a storage medium capable of being addressed and configured to execute one or more processors. For example, the elements may include elements such as software elements, object-oriented software elements, class elements, and task elements, processes, functions, attributes, procedures, subroutines, segments of a program code, drivers, firmware, microcode, circuits, data, databases, data structures, tables, arrays, and variables.

According to the various embodiments, each of the above-described elements (e.g., a module or a program) may include a singular entity or a plurality of entities. According to various embodiments, one or more of the above described elements or operations may be omitted, or one or more other elements or operations may be added. Alternatively, or additionally, a plurality of elements (e.g., modules or programs) may be integrated into one element. In this case, the integrated element may perform one or more functions of each of the plurality of elements in a manner the same as or similar to that performed by the corresponding element of the plurality of components before the integration. According to various embodiments, operations performed by a module, program, or other elements may be executed sequentially, in parallel, repeatedly, or heuristically, or one or more of the operations may be executed in a different order, or omitted, or one or more other operations may be added.

As is apparent from the above, according to various embodiments disclosed in this document, the presence of depression can be identified based on image data that is visualized input data. In addition, various effects that can be directly or indirectly identified through this document can be provided.

What is claimed is:

1. An apparatus for diagnosing a disease, comprising:
an acquisition module configured to acquire multi-modal data including at least two types of data among text data, speech data, and image data related to a test subject;
a preprocessing module configured to visualize data that is not the image data among the multi-modal data and output image datasets including the image data and the visualized data; and
a classification module configured to classify whether the test subject has a specified disease based on the image datasets.

2. The apparatus of claim 1, wherein the acquisition module separately extracts the image data and the speech data from a video data recorded during diagnosis of the test subject.

3. The apparatus of claim 2, wherein the video data includes at least one of a facial expression or a body movement of the test subject.

4. The apparatus of claim 2, wherein the text data includes at least one of text converted from the speech data extracted from the video data and text extracted from social networking services of the test subject.

5. The apparatus of claim 1, wherein, when the image data is color image data of two or more dimensions, the preprocessing module converts the image data into one-dimensional black and white image data.

6. The apparatus of claim 1, wherein the preprocessing module extracts an emotional keyword from the text data and visualizes the extracted emotional keyword as a word cloud.

7. The apparatus of claim 6, wherein the preprocessing module displays the extracted emotional keyword in the word cloud with a color and a size according to a frequency of appearance of the extracted emotional keyword and an emotional score of the extracted emotional keyword according to an emotional evaluation dictionary.

8. The apparatus of claim 1, wherein the preprocessing module applies a Mel spectrogram technique to the speech data to visualize the speech data.

9. The apparatus of claim 1, wherein, at least in an operation of training the classification module, the preprocessing module augments the image datasets using a data augmentation or a k-fold training technique when the number of the image datasets is less than a predetermined number.

10. The apparatus of claim 1, wherein the classification module includes a three-dimensional single network model based on convolution.

11. The apparatus of claim 1, which further acquires other data that is at least one of a heart rate, health data, and a life log of each of patients having the specified disease, and classify whether the test subject has the specified disease further based on the other data.

12. The apparatus of claim 1, wherein the specified disease includes at least one of depression, bipolar disorder, anxiety, depressive disorder, and anxiety disorder.

13. A method of diagnosing a disease, which is performed by at least one processor, the method comprising:
acquiring multi-modal data including at least two types of data among text data, speech data, and image data related to a test subject;
visualizing data that is not the image data among the multi-modal data;
outputting image datasets including the image data and the visualized data; and
classifying whether the test subject has a specified disease based on the image datasets.

14. The method of claim 13, wherein the acquiring of the multi-modal data includes separately extracting, from a video data recorded during diagnosis of the test subject, the image data including at least one of a facial expression or a body movement of the test subject and the speech data.

15. The method of claim 14, wherein the acquiring of the multi-modal data includes:
generating the text data by converting the speech data extracted from the video data into text; and
extracting the text data from social networking services of the test subject.

16. The method of claim 13, wherein the performing of preprocessing includes:
extracting an emotional keyword from the text data; and
visualizing the extracted emotional keyword as a word cloud.

17. The method of claim 13, wherein the performing of preprocessing includes applying a Mel spectrogram technique to the speech data to visualize the speech data.

18. The method of claim 13, wherein the performing of preprocessing includes augmenting the image datasets using a data augmentation or a k-fold training technique when the number of the image datasets is less than a predetermined number.

19. The method of claim 13, wherein the classifying of whether the test subject has the specified disease includes classifying whether the test subject has the specified disease through a three-dimensional single network model with a convolution structure.

20. An apparatus for diagnosing a disease, comprising:
a memory including at least one instruction; and
a processor functionally connected to the memory,
wherein when executed, the at least one instruction causes the processor to:
acquire multi-modal data including at least two types of data among text data, speech data, and image data related to a test subject;
visualize data that is not the image data among the multi-modal data, and output an image dataset including the image data and the visualized data; and
classify whether the test subject has a specified disease based on the image dataset.
